(19) United States
(12) Patent Application Publication
(10) Pub. No.: US 2020/0367749 A1
(43) Pub. Date: Nov. 26, 2020

(54) IMPLANTABLE WIRELESS NETWORK OF DISTRIBUTED MICROSCALE SENSORS

(71) Applicants: Brown University, Providence, RI (US); University of California, San Diego, La Jolla, CA (US)

(72) Inventors: Arto NURMIKKO, Providence, RI (US); Farah LAIWALLA, Barrington, RI (US); Jihun LEE, Providence, RI (US); Vincent Wingching LEUNG, San Diego, CA (US); Patrick MERCIER, San Diego, CA (US); Lawrence Ernest LARSON, Providence, RI (US); Peter Michael ASBECK, Del Mar, CA (US)

(21) Appl. No.: 16/879,216

(22) Filed: May 20, 2020

Related U.S. Application Data
(60) Provisional application No. 62/850,202, filed on May 20, 2019.

(51) Int. Cl. A61B 5/00; H04B 1/3827; H04J 3/02; A61B 5/04

(52) U.S. Cl. CPC A61B 5/0031; H04B 1/385; H04J 3/02; A61B 5/0024; H04W 84/18; A61B 5/0022; A61B 5/7267; A61B 5/7228; A61B 5/4836; A61B 5/04001

(57) ABSTRACT

A system includes an ensemble of wirelessly networked intracranial implants, and a compact external epidermal wearable skin patch radio frequency (RF) transceiver and data processing hub, the network of intracranial implants wirelessly linked to the skin patch RF transceiver and data processing hub.

### CROSS REFERENCE TO RELATED APPLICATIONS

This application claims benefit from U.S. Provisional Patent Application Ser. No. 62/850,202, filed May 20, 2019, which is incorporated by reference in its entirety.

### STATEMENT REGARDING GOVERNMENT INTEREST

This invention was made with government support under SPAWAR grant no. N66001-17-C-4013 awarded by ONR. The government has certain rights in the invention.

### BACKGROUND OF THE INVENTION

The present invention relates generally to sensors, and more particularly to an implantable wireless network of distributed microscale sensors.

In general, a vastly enhanced capability to bi-directionally interface with cortical microcircuits in a clinically viable way is the ultimate aspiration in neuroengineering. This necessitates a paradigm shift in neural interface system design beyond current bulky, monolithic constructs which are challenging to scale past 100-200 channels due to anatomic and engineering design constraints.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention.

Its sole purpose is to present some concepts of the invention in a simplified form as a prelude to the more detailed description that is presented later.

In general, in one aspect, the invention features a system including an ensemble of wirelessly networked intracranial implants, and a compact external epidermal wearable skin patch radio frequency (RF) transceiver and data processing hub, the network of intracranial implants wirelessly linked to the skin patch RF transceiver and data processing hub.

In another aspect, the invention features a system including a spatially-distributed network of wireless microscale implantable sensors capable of recording and electrical stimulation, a compact wearable skin patch including an external wireless hub and a neuro-computational processor, the spatially-distributed network of wireless microscale implantable sensors wirelessly linked to the compact wearable skin patch, and a remote server, the compact wearable skin patch wirelessly linked to the remote server.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is block diagram of an exemplary system.

FIG. 2(a) illustrates a schematic overview of an ultra-compact, low-noise AFE.

FIG. 2(b) illustrates a synchronized bidirectional wireless TDMA network.

FIG. 2(c) illustrates Tx and relay coil geometries and power-transfer efficiencies for a sample-coil-system.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The present invention is directed towards a neural interface system relying on a spatially-distributed network of wireless microscale implantable sensors that offers a highly scalable, robust and adaptive architecture for next-generation neural interfaces. More specifically, the present invention includes a wireless network of sub-mm, untethered, individually addressable, fully wireless microscale sensors (referred to as “neurograins”), in the context of an epicortical implant. Individual neurograin chiplets integrate a ˜1 GHz wireless link for energy harvesting and telemetry with analog and digital electronics for neural signal amplification, on-chip storage, and networked communications via a Time-Division Multiple Access (TDMA) protocol. Each neurograin forms a completely self-contained single channel of neural access and is implantable after post-process atomic layer deposition of thin-film (e.g., 100 nm thick) barriers for hermetic sealing. Ensembles of implantable neurograins form a fully wireless cortico-computer communication network (utilizing their unique device IDs). The implanted network is coordinated by a compact external epidermal skin patch radio frequency (RF) transceiver and data processing hub, which is implemented as a wearable module in order to be compatible with clinical implant considerations.

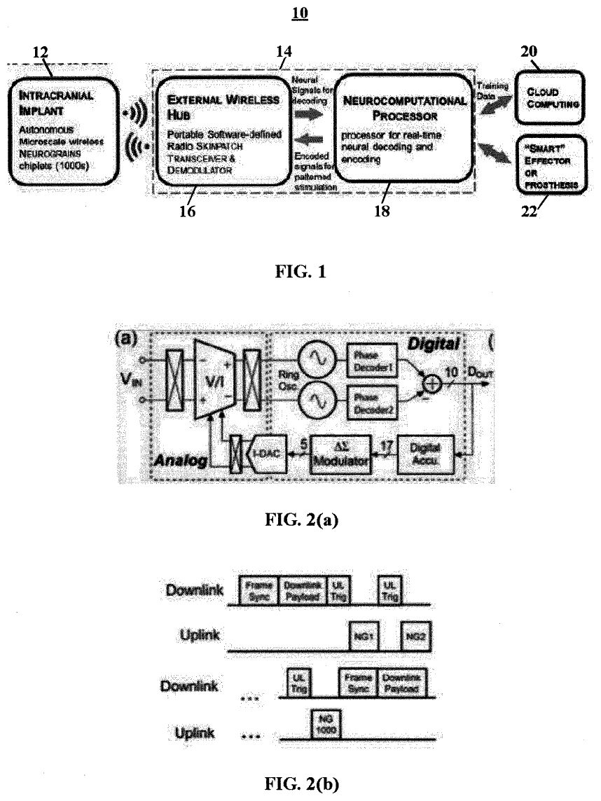

As shown in FIG. 1, an exemplary system 10 includes an ensemble of wirelessly networked intracranial implants 12 wirelessly linked to a compact external epidermal wearable skin patch 14. The compact external epidermal wearable skin patch 14 includes radio frequency (RF) transceiver 16 and data processing hub 18. The data processing hub 18 may be communicatively linked to one or more cloud computing servers 20 and/or smart effector and/or prosthesis 22.

Each of the intracranial implants 12 includes a wireless microscale implantable sensor that is of autonomous sub-millimeter microscale size. Each implantable sensor integrates a wireless link operating near 1 GHz frequency for energy harvesting and telemetry with analog and digital electronics for neural signal amplification, on-chip storage, and networked communications using, for example, a time-division multiple access (TDMA) protocol. Each sensor 12 is capable of either or both neural signal recording and microscale electrical stimulation of neuronal targets.

The radio frequency (RF) transceiver 16 and data processing hub 18 include a field-programmable-gate-array (FPGA) back-end configured to provide real-time wireless demodulation and neural decoding/encoding for closed-loop control.

The one or more cloud computing servers 20 may be configured for model optimization and machine learning techniques.

In summary, scalability and implantability are important drivers dictating the system 10 architecture. As such, the system 10 constitutes ensembles of implantable, sub-millimeter, individually addressable, microelectronic chiplets (referred to herein as “neurograins”). Each neurograin is designed as a self-contained, hermetically sealed module measuring, for example, 500 μm×500 μm×35 μm, and powered through transcutaneous wireless power delivery via near-field inductive coupling at approximately 1 GHz. This activates on-chip electronics for neural signal transduction (ECoG signals with up to 500 Hz bandwidth in this epi-cortical application), on-chip memory storage and subsequent uplink telemetry via RF backscattering at 10 Mbps in accordance with the implemented time-domain multiple access (TDMA) network protocol. The network communication protocol is designed to accommodate up to 1000 channels of broadband ECoG data, but extendable via parallelization and other techniques to 10-100× higher channel counts with compatible hardware changes. Neurograin microdevices can be individually or collectively implanted (latter e.g., an ECoG grid or peripheral nerve cuff electrode) into selected areas of the brain or peripheral nervous system (PNS), thereby forming untethered, broadband, bidirectional neural interfacing elements usable for a variety of diagnostic and therapeutic neural applications.

At the core of the neurograin implant 12 is an Application-Specific Integrated Circuit (ASIC), designed in 65 nm mixed-signal low-power process. This 0.25 mm² chip integrates analog, digital and RF circuitry allowing for autonomous operation, and can thus be deployed independently into the brain. Networked chip telecommunications are coordinated by a wearable external software-defined-radio (SDR). The latter has a field-programmable-gate-array (FPGA) back-end, which is utilized to provide real-time wireless demodulation, as well as neural decoding/encoding for closed-loop control. This neurocomputational processor is implemented to seamlessly access resources such as cloud computing for model optimization such as example, machine learning techniques.

The neurograin chiplets 12 implement an on-chip multi-turn microcoil along with RF rectifiers and backscatter modulators.

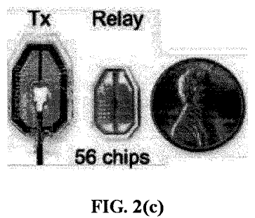

Sensing neural activity with high-fidelity is a critical requirement for the wireless neurograin sensors. The present invention provides a self-standing, DC-coupled AFE with a merged amp-ADC architecture. By combining a chopping V/I converter, a VCO-based ADC and a mixed-signal differential electrode offset (DEO) canceling servo loop (see FIG. 2(a)), system 10 eliminates capacitor use, leading both to large area savings and elimination of kT/C noise. The analog front end (AFE) is designed for EcoG signals, with a bandwidth of 500 Hz and a dynamic range of ±1 mV (where the latter is adequate for differential signals from neurograin electrodes with an inter-electrode electrode spacing of ˜100 μm). The DC-offset cancellation range is ±50 mV, with 3.2 μW of power consumption from a 0.6 V supply. Input-referred noise is 2.2 μVrms over 500 Hz bandwidth for this implementation, which occupies 0.01 mm².

Neurograin chiplets have a unique on-chip ID, which is derived from utilizing CMOS process variations. This ID (discoverable during chip calibration) is used to queue neurograin nodes for a TDMA telecommunication network. The uplink telemetry uses Binary Phase Shift Keying (BPSK)-modulated RF backscatter at 10 Mbps, while the downlink employs amplitude-shift-keying based pulse-width-modulation (ASK-PWM) at 1 Mbps. The same wireless link is used for both power and telecoms, and the full network design relies on synchronized bidirectional communication for efficient link bandwidth usage (see FIG. 2(b)), accommodating up to 1000 broadband ECoG neurograins on a single RF channel. We have also implemented in parallel a non-synchronized, periodic, packetized uplink-only TDMA network, which utilizes link bandwidth less efficiently and may suffer from occasional data collisions, but none the less provides an effective way to rapidly test small networks with fewer than 100 nodes such as described herein.

The neurograin implants 12 are wirelessly powered in the near-field inductive coupling regime at ˜1 GHz. The power budget for an individual neurograin is ˜40 μW, and this must be harvested across a transcranial distance of 1 cm. The present invention accomplishes this by the introduction of a relay coil which has moderate coupling both with the external Tx and the neurograin microRx coils, thus increasing the efficiency of wireless power transfer at least 50× over a standard 2-coil approach. Furthermore, both the Tx and relay coils are designed with a double coil layout architecture, which creates a uniform magnetic field over a large plane enabling us to capture a large number of spatially distributed neurograin microdevices 12 with a single Tx-relay pair. One example is a 4-quadrant coil design covering a 2 cm×2 cm area for a 1000 channel human implant. Coil geometries may be further adapted to accommodate anatomical variations.

Chronic biocompatibility of the neural implant is a consideration of utmost importance for clinical viability. A robust physical electrode-tissue interface is a major aspect of this challenge along with hermetic packaging of the implant. We utilize standard post-process microfabrication techniques for patterned deposition of gold on top of the fabricated chiplet pads to form neurograin recording electrodes with impedances in the 100 kΩ range. We have also developed techniques for ALD-based stacked multilayer conformal coatings for neurograin hermetic packaging. The overall thickness profile of the packaging material is 100 nm, and packaged chiplets have been tested in an accelerated aging testbed with demonstrated viability well over 10 years at this time.

In one implementation, the present invention uses a compact SDR platform consisting of an Analog Devices AD9361 RF transceiver and a Zynq UltraScale+ system-on-chip (SoC) as the external telecommunications hub (see FIG. 2(c)). The SDR is used to provide an RF transmission tone at 915 MHz (which is amplified using an IC-based power amplifier) for powering neurograins. The receive pathway on the SDR is fed by a duplexer (D5DA942M5K2S2) allowing for isolation of the BPSK-modulated data lobe. The SDR receiver front-end provides signal amplification, I/Q demodulation, and digitization at 45 MSa/s. The digitized IQ data may then either be ported to a PC for offline processing(in MATLAB®/Simulink®), or processed in real time using the Zyng™ FPGA (implementing HDL-optimized versions of the Simulink phase and sample timing recovery algorithms prior to BPSK demodulation).

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A system comprising:
an ensemble of wirelessly networked intracranial implants; and
a compact external epidermal wearable skin patch radio frequency (RF) transceiver and data processing hub, the network of intracranial implants wirelessly linked to the skin patch RF transceiver and data processing hub.

2. The system of claim 1 wherein each of the intracranial implants comprises a wireless microscale implantable sensor.

3. The system of claim 2 wherein each wireless autonomous sub-millimeter size microscale implantable sensor integrates wireless link operating near 1 GHz frequency for energy harvesting and telemetry with analog and digital electronics for neural signal amplification, on-chip storage, and networked communications using a time-division multiple access (TDMA) protocol.

4. The system of claim 3 wherein each sensor is capable of either or both neural signal recording and microscale electrical stimulation of neuronal targets.

5. The system of claim 3 wherein each wireless microscale implantable sensor comprises a unique on-chip ID configured for TDMA communications.

6. The system of claim 1 wherein the RF transceiver and data processing hub comprises a field-programmable-gate-array (FPGA) back-end configured to provide real-time wireless demodulation and neural decoding/encoding for closed-loop control.

7. The system of claim 6 wherein the RF transceiver and data processing hub wireless communication with one or more remote servers.

8. The system of claim 7 wherein the one or more remote servers is configured for cloud computing for model optimization and machine learning techniques.

9. A system comprising:
a spatially-distributed network of wireless microscale implantable sensors capable of recording and electrical stimulation;
a compact wearable skin patch comprising an external wireless hub and a neuro-computational processor, the spatially-distributed network of wireless microscale implantable sensors wirelessly linked to the compact wearable skin patch; and
a remote server, the compact wearable skin patch wirelessly linked to the remote server.

10. The system of claim 9 wherein each of the wireless microscale implantable sensors comprises a 1 GHz wireless link for energy harvesting and telemetry with analog and digital electronics for neural signal amplification, on-chip storage, and networked communications.

11. The system of claim 10 wherein the networked communications utilizes time-division multiple access (TDMA) protocol.

12. The system of claim 11 wherein each of the wireless microscale implantable sensors comprises a unique on-chip ID configured for TDMA communications.

13. The system of claim 9 wherein the external wireless hub comprises:
a transceiver; and
a demodular.

14. The system of claim 13 wherein the neuro-computational processor is configured for real-time neural decoding and encoding.

15. The system of claim 9 wherein the remote server is configured for cloud computing.

16. The system of claim 15 wherein the cloud computing comprises model optimization and machine learning techniques.
